(19) United States
(12) Patent Application Publication
(10) Pub. No.: US 2008/0086128 A1
(43) Pub. Date: Apr. 10, 2008

(54) Method and apparatus for treatment of scoliosis

(76) Inventor: David Warren Lewis, Charlottesville, VA (US)

Correspondence Address: Leo J. Aubel, 111 Rivershire Lane, Lincolnshire, IL, 60069, US

(21) Appl. No.: 11/516,968

(22) Filed: Sep. 7, 2006

(51) Int. Cl. A61F 2/30

(52) U.S. Cl. 606/61

(57) ABSTRACT

Non-surgical treatments for idiopathic scoliosis include muscle stimulation therapy, chiropractic care, and the application of a variety of braces (orthotics). Surgical intervention frequently employs rigid metallic braces that prevent further flexing of the spine where applied. This invention allows flexing of the spine with long term correction of the scoliosis by application of small variable forces to supplant and counter the unbalance of the pertinent muscles. This invention may be applied to other spine problems in addition to idiopathic scoliosis as this invention permits and accommodates flexing of the spine and simultaneously supplies correcting and straightening forces.

### FIELD OF THE INVENTION

The field of this invention bears directly on the subject of idiopathic scoliosis offering a new approach to the treatment thereof. This invention offers an alternative to other treatments of spinal deformities and is suitable for all ages of human beings due to the inherent flexibilities that are incorporated into the concept as contrasted with the more common employ of rigid wires, braces, and fixtures. Initial work in this subject was published in the “Journal of Bone and Joint Surgery” VOLUME 44-A, No. 4, June 1962 by Paul R. Harrington, whose procedure carries the name Harrington as in Harrington Rod. An alternative to this work was presented as U.S. Pat. No. 4,085,744 by Lewis, et al. Apr. 25, 1978. This invention evolved from said U.S. Pat. No. 4,085,744 that addressed the problem of providing a series of forces through attachment to many vertebral bodies. These forces could be adjusted through surgical means. This invention supercedes the need for additional surgical intervention as once this invention is in place, force levels provided by it automatically are controlled as the spine format corrects itself.

### BACKGROUND OF THE INVENTION

The Scoliosis Research Society, dedicated to the education, research, and treatment of spinal deformity notes that idiopathic scoliosis occurs in infants, juveniles, and adolescents. As by its definition, the cause of idiopathic scoliosis is not known and therefore requires careful, quantitative, and repeated observations and measurements. The adolescent type, defined from 10-18 years of age, is the most common and represents about 80% of this type of scoliosis.

Treatment for scoliosis ranges from a series of observations over time in infants to surgery in severe cases. Many infants, especially boys, grow out of the scoliosis hence close vigil should be the “treatment” initially. Some kinds of chiropractic manipulations and other stretching and motion exercises are suggested by some practitioners as a means for overcoming the unbalanced forces in the musculature and tendons. Juvenile idiopathic scoliosis (3-9 year olds) may rapidly progress especially in children over the age of five and may require comprehensive orthotic (brace) management. Surgery is ultimately indicated if the undesirable curve of the spine is unable to be controlled by orthotic means.

Surgery may result in some foreshortening of the spine but is thought to be more desirable than allowing the curvature to increase which may cause other serious physiological problems. Frequently, surgery involves the incorporation of metallic bracing and/or fusion of bones that result in rigidity of that portion of the spine. This therefore limits certain motions and flexing of the spine and future growth of body elements. The alternative to this rigid bracing and fusion is the subject of this invention.

It should be noted that the scoliosis condition does not generally involve lower back pain which is the subject of many patents and patent applications. The lack of lower back pain is significant as the condition may degenerate slowly and not be noticeable. There are many patents directed to the treatment and alleviating of lower back pain that are not germane to the subject of this patent. Such lower back pain alleviating patents involve the application of forces and devices that are contra-indicated for the treatment of idiopathic scoliosis. Such lower back pain patented devices involve severe manipulation of sectors of the spine through application of large forces. This invention, not dealing with lower back pain, involves the application of small variable forces that continue to decrease as the device resolves the unbalanced musculature.

This invention involves surgical intervention with the insertion of one or two different configurations of this device. One configuration of the device is attached to the pedicles of two separate vertebrae. The pedicles are singled out as having much strength but on some applications, an alternate fastening of the device will be to the transverse processes. The device, when attached to either the pedicles or the transverse processes, provides a variable force, depending upon the initial stretch or preload of the spring and of the spring rate designed into the device, and the amount of flexion resulting from rotation of the spinal column. It is this combination of flexibility and variable force that distinguishes this unique device from all other surgical implants onto the spine.

This device, in one configuration, provides a tensile force, even small in value, which supplements the muscles that have been weakened or otherwise have been overcome by other unbalanced muscles acting in opposition. The long term effects of this device provide small forces that are relieved, as the undesirable spine contour is reduced, due to the diminishing of the spring force composing one main element of the invention. This device may be employed singly or multiply as determined by the practitioner.

A second configuration of this device may be identified principally as a compression element. This is configured so as to increase the distance between pedicles or force apart the pedicles when attached to the two ends of the device. Note, the two pedicles selected for application of this device may be of immediately adjacent vertebrae, or not, depending upon the initial degree of curvature of the spine. With modifications to the attachment means, this same general compression configuration may be attached to transverse processes rather than pedicles. The treatment decided upon by the surgeon will determine which vertebral bodies are selected and which sections of said bodies are chosen. As before for the tensile device, the flexibility of this device stems from its spring rate and the amount of motion exhibited by the patient. As the force levels employed when using this invention are purposely designed to be small, attachment to transverse processes is not as risky as are the more rigid metallic devices attributed to the Harrington concept.

Scoliosis is a unique form of spinal degradation. It is not to be confused with treatments directed to lower back pain such as Patent No.: US 2005/0171543 A1 which addresses the problem of spine stabilization associated with injuries and fractures. Said patent specifically lists “a multitude of surgical/anatomical settings, including specifically long bone applications involving the femur, tibia, fibula, ulna, and or humerus.” Said patent is directed to “lower back pain” and “spine stabilization” and to “balance the loads applied to the spine”. The scoliosis treatment employing this new patent is not for the relief of pain (typically there is no pain reported by the patent) nor is it to stabilize the spine (the spine is stable however it is not properly aligned) nor is it broken or fractured (it is misconfigured from unbalanced muscles and tendon tensions). The Patent No.: US 2005/0171543 A1 has to provide substantial forces to maintain proper alignment for the efficacious healing that is associated with lower back pain and spinal stabilization. Without substantial forces, said patent could not maintain the stabilization necessary to control the proper contact alignment and stabilization. This new patent concept makes use of long-term small forces to compensate for unbalanced musculature and tendons and does not provide for alignment, does not maintain contact of bone fractures, doe not control the stabilization of the spine for lifting heavy loads, and is not directed to the relief of lower back pain.

The two major configurations of the devices of this new patent described above will be used singly or in combination depending upon the degree of curvature and location of the primary curvature of the spine which has resulted from a long term unbalance of the muscles and tendons not from any accidental fracture or accidental spinal overload. It may be necessary to employ more than one of either or both device configurations and with different spring rates incorporated into the devices. One combination of these two configurations will be illustrated in the drawings and description which follows.

The philosophical difference in using the presently employed rigid bracing implants and the flexible devices of this invention will require planning by the surgeon. In addition, with this new invention, patients will have to be taught to restrain themselves initially as they will retain much of their initial spinal flexibility. As the forces of this device continue to interact with the forces of the patient's own muscles, the spine will slowly become more normal in contour. Simultaneously, the spring forces, even though they are of the order of one or two pounds, in this device will decrease as the muscles that have been overpowering their opposing and adjacent muscles compensate for their associated forces.

It is known, physiologically, that even a small force applied to a muscle will ultimately cause yielding, stretching, and relaxation and an elongation of the muscle. And so the application of this invention will cause redistribution of the normal muscle activities that have been causing the spine curvature to initiate and to progress. This physiological phenomenon is the basis of this patent and differentiates it from the more normal surgical procedures and patent devices which are applied to stabilize and control lower back pain. These referenced surgical procedures are applied to immediately change the orientation of the elements of the back to affix the configuration resulting from fracture or accidental overload. This patent is concerned with the reorientation of the elements of the back over an extended period of time even without any indication of lower back pain.

### BRIEF DESCRIPTION OF THE DRAWINGS

FIG. 1 is a cross section of one form of this invention generally affording a tensile force when applied to the pedicles of the spine, showing the main elements including the casing 1, the tension spring 2, the movable head 3, the base head 4, and the holes 5 through which restraining screws (not shown) will be placed, said restraining screws will transmit the forces F1 and F2 to the pedicles that they are screwed to.

FIG. 2 is a cross section of one form of this invention illustrating the means for applying a compressive force when affixed to the spine through the vertebral bodies, showing the casing 11, the compression spring 12, the movable head 13, the base head 14, and the holes 15 through which restraining screws (not shown) will be placed, said restraining screws will transmit the forces F1 and F2 to the pedicles to which they are affixed.

FIG. 3 illustrates a portion of the human spine 30, a compression configuration device 31 of this invention, a second compression configuration device 32, and an extension or extraction configuration device 33 of this invention. In FIG. 3, one transverse process 34 in identified as one of many illustrated in the figure. The compression configuration and extraction configuration devices of this invention are pictured as being affixed to the respective transverse processes. However, the first choice by the surgeon for affixing the devices of this invention will be using the pedicles as they are generally stronger than the transverse processes even though the devices of this patent are not likely to exert forces exceeding a few pounds.

FIG. 4 shows a cross section of another embodiment of this invention with the casing 50, the compression spring 51, the base head 52, the movable head 53, and a special flexible sealed bladder 54. One special variation or form of this invention can be recognized by the removal of the spring 51 of FIG. 4 and having the bladder 54 pressurized with air or gas. In this particular form, with the spring 51 removed, the casing 50 can be made shorter so that the device now functions as an extension rather than as a compression device. The spring rate of the device is determined by the cross sectional area of the bladder 54, its length and the initial pressure in the bladder before it is moved from the position as illustrated.

FIG. 5 illustrates another extraction form of this invention employing linear springs 60 with the movable head 62 pressed nearly into or against the stop 64 of the base head 61. The linear spring may be made from rubber or one of several different forms of plastic each of which must be compatible with the human body. The forces F1 and F2 would be applied to the pedicles through screws (not shown) inserted through the respective holes in the base head 61 and the movable head 62.

FIG. 6 depicts yet another embodiment of this invention as a series of interconnected bellows that may be designed such that the embodiment is in either the compression configuration or the extraction configuration mode or both. The spring rate for this embodiment is determined by the material thickness, the material type, the inner and outer diameters of the individual bellow elements, and the number of bellow elements.

FIG. 7 shows the cross section of yet another embodiment of this invention with the base head 72, movable head 73, upper bladder 74, lower bladder 71, and case 70. The effective spring rate of this embodiment is determined by the relative cross-sections of the upper and lower bladders and the precharge pressure of the air or gas in each bladder.

FIG. 8 is a cross section of another embodiment of this invention, which makes use of “wave springs”. Wave springs offer certain design advantages over the more conventional helical springs including stability and smallness of overall length as compared with helical springs. This particular illustration offers one design that can function as either an extraction or compression configuration when the individual circumferential lines of contact of the wave springs are welded or otherwise fastened together.

### DETAILED DESCRIPTION OF THE INVENTION

Certain physical deformities become apparent in the skeletal shape of the human being that can be traced to unbalances in the musculature and the tendon fixations. One classification of these deformities is noted as idiopathic scoliosis. This invention is directed to overcoming several such physical deformities of the spine including idiopathic scoliosis. Said physical deformities are to be distinguished from fractures, over stressed elements of the spine from severe exercises, accidents, or accidental overloads, dynamic spine destabilizing actions, and other episodes generally resulting in what is classified as lower back pain. Idiopathic scoliosis is not generally associated with back pain or fractures or other accidental occurrences but it does have follow-on physiological ramifications with lungs, heart, and other body organs.

This invention introduces one force or a set of forces that oppose the musculature unbalances that, with time, have caused the skeletal shape to be distorted. This said distortion causes other physiological upsets to the human anatomy that, ultimately, may be so severe as to threaten the life of the person. If the body does not compensate for these muscle unbalances during the early growth years, certain orthotic treatments may be attempted whose purpose is to halt or stimulate other muscle counterbalances. Some chiropractic approaches involving stretching and particular body motions may also be directed toward compensating for the muscle unbalances that are producing the skeletal distortions.

If normal growth does not overcome the undesirable muscle and tendon unbalances and if orthotic treatments are not successful then surgery may be necessary. In the past, the surgical approach involved either vertebral modifications including fusion or the implantation of metallic rods and braces or some combination of the two. These rods and braces, when affixed to the spine are generally rigid and therefore cause some restriction of motion of the body. Further, these implanted rods and braces are subject to revisions if they are applied to a youngster who is still growing and whose restraints from the rods and braces are in themselves causing improper growth. As previously noted, these metallic physical restraining elements have collectively been identified as the Harrington approach and do straighten, to some degree, the extreme curvatures of the spine with an incumbent stiffening and restraint on spine flexibility.

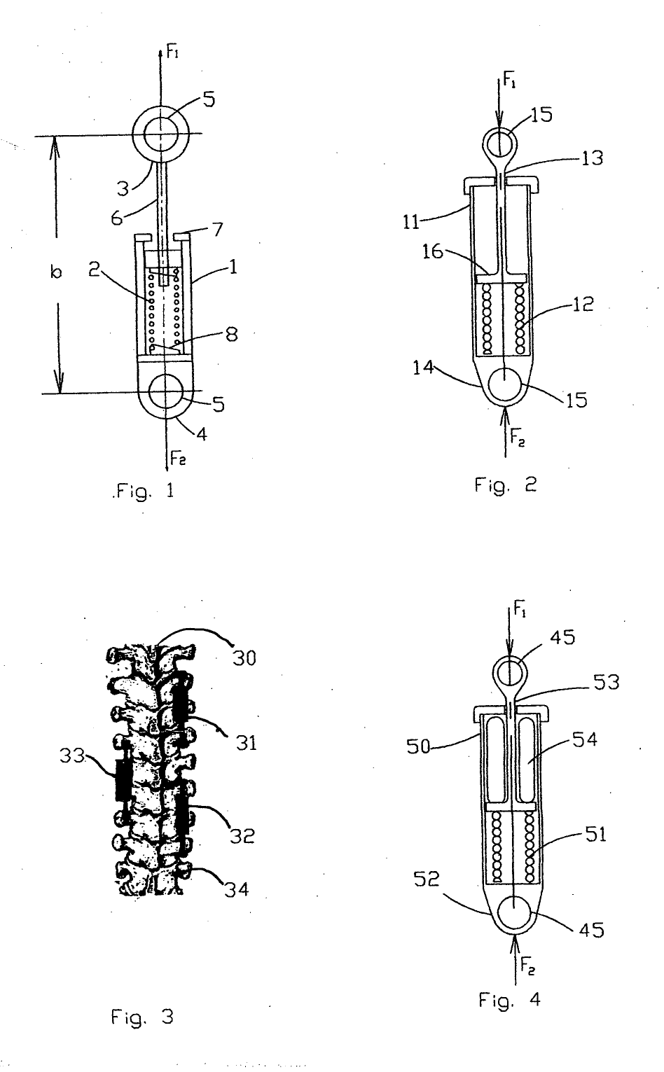

This invention provides the means for supplying variable forces that are self-adjusting as the body flexes and are directed in a manner to oppose the unbalanced musculature. The cross section of one embodiment of this invention is given in FIG. 1 whose elements can be understood to move as forces, F1 and F2 are applied to the opposite ends of the device through elements, typically screws (not shown), inserted in the holes 5 of the movable head 3 and the base head 4. As shown, the forces F1 and F2, being equal in value and oppositely directed have caused the spring 2 inside the case 1 to extend and thereby cause stresses in the spring that balance said forces F1 and F2. The value of the force F1 or F2 can be calculated as it is related to the other defining properties of the spring. For a spring constructed from wire with a circular cross section, the fundamental equation given in the book “Design Of Machine Elements” by M. F. Spotts © 1978 by Prentice-Hall, Inc. is

Ss=Ks×2×F×c³/(π×R²)

in which Ss=shearing stress in the material in the units of pounds per square inch (abbreviated as psi), Ks=stress multiplication factor, F=force in pounds, c=2×R/d, the spring index, d=wire diameter in inches, π=pi or approximately 3.14159, and R=mean radius of the helix of the spring in inches. The spring rate k=d⁴×G/(64×R³×N), in which k is given as the pounds load for a unit deflection of the spring, G=modulus of elasticity of the spring material in shear (in psi), and N=number of active coils of the spring. With the above equations one may determine the proper elements of the spring to arrive at the desired overall size, spring rate, and maximum design stress for a given material selection. And for the selected one to two pound range of forces desired for this device, the size may be made very small allowing for insertion into the spine area with relative ease and with little external display or enlargement and distention of the covering body flesh and skin.

As pictured in FIG. 1, F1 and F2 have caused the spring 2 to be extended almost to a limit as the movable head 3 has come close to moving the spring end cap 9 into bearing against the retainer stop 7. This embodiment may be designed to yield a variable force up to some specified value such as one pound or two pounds. As the length noted by “b” between the centers of the holes 5 decreases, the value of the forces F1 and F2 will decrease. It is this variation in the value of the forces F1 and F2, as the spine flexes and the distance between pedicles changes, that makes this invention unique from the alternate uses of rigid braces and the fusion process. Further, this variation in the value of the force in this device accommodates to the variation of the forces in the muscles acting on the spine. It is important to note that the small forces supplied by the embodiment of this patent act continuously and hence compensate for the unbalanced muscular forces that are causing the undesirable curvature of the spine. Other patents that may be cited for stabilizing of a spine related to lower back pain must be extremely large when referenced to fractures and similarly over-stressed muscles and tendons. Stating again, this patent is not directed to situations producing lower back pain nor is it directed to producing large forces that might lead to lower back pain.

In FIG. 1, the movable head 3 has a threaded stock 6 that may be rotated relative to the end cap element 9 to which is affixed one end of the spring 2. This feature, simply by rotating the movable head 3 relative to the base head 4, affords an adjustment to the overall length of the embodiment for precise location and force level control in affixing the device to the pedicles during the surgical procedure. By changing the overall length, the position of each of the pedicle screws, not shown, when inserted through the holes 5 of the movable head 3 and the base head 4 may be properly aligned to the most structurally sound portions of the pedicles.

FIG. 2 is a cross section of another embodiment of this invention configured so as to produce an extraction or extensive force when applied to the spine through attachment to the pedicles or transverse processes. The casing 11 incorporates the base head 14 with a hole 15, which affords the means for applying the force F2 through an element, typically a screw (not shown), inserted in the hole 15. In this same FIG. 2, an equal force F1 directed opposite the force F2 just cited, is shown acting through a hole 15 of the extension head 13 and an integral piston 16, which acts on the spring 12. In this illustration, the spring 12 is shown compressed to its limit meaning that each helical coil is pressed against its adjacent coil. As can be understood by anyone versed in the field of solid mechanics, the forces F1 and F2 pictured in FIG. 2 will be acting in an opposite direction, via screws (not pictured but acting through the holes 15), onto the spine element to which the screws are attached. The resulting action of the pictured form of this invention is to extract or cause additional separation of the bone elements, the vertebral bodies, of the spine to which the device is attached. As described before, incorporating this spring in the device allows for the flexing of the spine and simultaneously provides a variable force which act on those muscles to which is attributed the scoliosis.

### General Overview

As an example in selecting parameters associated with the simple spring design of FIG. 1, assume a #21 wire with the diameter d=0.0317 inch. Assume further a helix count N=10, the nominal coil diameter D=0.25 inch, which equals 2×R, so that from initial touching of the coils to the overall extension of 0.633 inch, the maximum force can be calculated as F=2 pounds, and the maximum shear stress can be calculated as 43,226 psi. The maximum elongation of the spring yielding the 2.0 pound load will be 0.216 inch from which may be calculated the spring constant k=2/0.216 or k=9.26 pounds per inch. This is just an example illustrating one set of arbitrarily selected parameters for the embodiment of this invention and applying the equations given previously.

Even with a maximum 2.0 pound force, produced as noted by the parameters selected above, acting on a set of muscles, the muscles will stretch and thereby allow the spring to contract in overall length and the associated force acting through this invention to become smaller. Note that as muscles flex, this invention will accommodate the flexing motion by automatically changing the force produced by this device. And as the spine continues to return to the more proper natural curvature, the force(s) of the devices of this invention, assuming several are used, will be reduced and ultimately may become zero. The employment of the embodiments of this patent is to correct the muscular unbalances that have caused an improper alignment of the spine. This patent is in contrast to patents directed to problems identified as “lower back pain” or “spinal stabilization problems” as scoliosis patients, generally, do not have lower back pain or instabilities of the spine. Said scoliosis patients can perform everyday functions quite satisfactorily without pain or spine instabilities.

To amplify the significance of the changing forces that this invention affords the surgeon, imagine that the portion of the spine illustrated in FIG. 3 has a curvature that may be thought of as a backward “C” or ). With the extraction configuration 33 of this device pictured on the left side of the spine in FIG. 3, the tendency will be to “open” the backward “C”. Simultaneously, the compression configuration devices 31 and 32 of this illustration pictured on the right side of the spine in FIG. 3 will be acting in a manner to also open the backward “C”. The proper selection and number of extraction and compression devices will be determined by the surgeon depending upon the degree of curvature that needs to be corrected. As each of the devices of this invention are springs yielding variable forces, as the spine in FIG. 3 becomes more straight, the values of the forces in the devices will decrease. And as can be understood by this self-accommodating combination of forces, the spine may be flexed during regular body activities and when returned to the more normal attitude, the forces in the devices will return to their more normal force values. Contrast this action with the use of rigid braces and wires, which will not allow flexing nor will they correct the spine curvature over time.

As illustrated above, the sizes of the forces, being as they act over long periods of time, need not be large. A one or two pound force will have a large influence on several muscles over extended periods of time. These small forces imply that the springs may be made from materials other than stainless steel or other metals that are compatible with body fluids. Certain plastics, which are materially compatible with the human body fluids, when formed as springs can yield a one or two pound force.

For anyone versed in the art of mechanics, FIG. 4 can be visualized as having no mechanical spring 51 in the figure but instead having solely a flexible bladder 54 filled with air or compressed gas. As the movable head 53 is extended outward from the casing 50, in the the direction symbolized by the vector representing the force F2, the pressure of the air or gas in the flexible bladder will increase thereby causing the configuration to act as a compression configuration device. Alternatively, if a flexible bladder replaces the spring 51 of FIG. 4, the unit will act as an extraction configuration device. Further, the use of a flexible bladder, with or without a mechanical spring, will contribute damping in the operation of the devices. Said damping may be desirable especially for very active people as it will minimize any undesired “bouncing” of the spring.

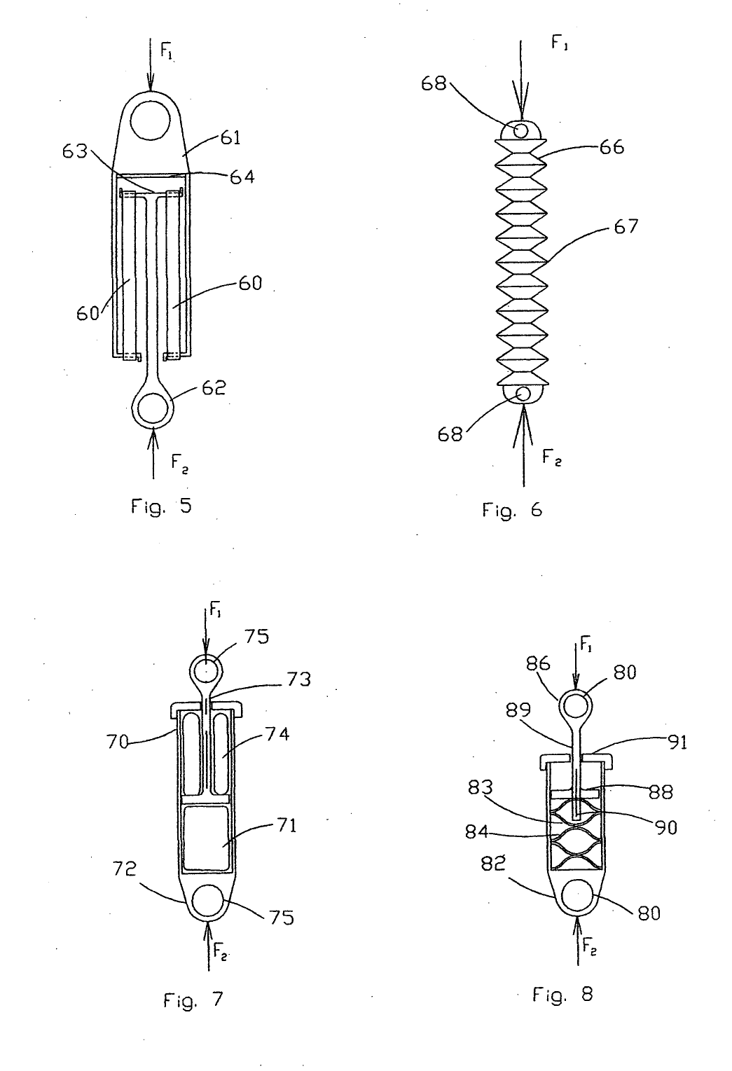

Another embodiment of this invention is given in FIG. 5 shown with the flexible elements 60 stretched by forces F1 and F2 acting, respectively, on the movable head 62 and the base head 61. This embodiment offers the advantage of the spring rate being easily modified by changing the size of the flexing elements 60. As pictured in the illustration, the flexing elements 60 have been stretched or extended almost to the limits of the design as the extreme end 63 of the movable head has nearly reached the cavity end 64 of the base head 61. By the selection of the material of the flexing elements 60, inherent damping can be determined for the device. As noted before, damping may be very desirable when this invention is applied to certain active human beings. A side view of this embodiment would show restraining guides, not pictured, to maintain planar alignment of the elements 61 and 62. Anyone versed in the art of design can easily visualize how this design can be “inverted” in operation to yield a compression configuration device instead of the extraction form as pictured.

An additional embodiment of this invention, illustrated in FIG. 6, is through a series of interconnected bellows. Anyone versed in the art of mechanical design can understand how it may be created such that it acts in either the compression configuration or the extraction configuration mode or both. The overall spring rate for this embodiment is determined by the material thickness and type of material from which the bellows are formed, the inner and outer diameters of the individual bellows elements, and the number of bellows elements.

The attachment means to the spinal column pedicles will be by screws (not shown) through the holes 68, of FIG. 6. FIG. 6 illustrates the external forces F1 and F2 acting on the device to make it shorter thereby, when attached to the pedicles, this device will be acting in an extraction configuration. As described above, the means for attachment to the pedicles will be through screws but attachment to transverse processes may be accomplished by wires or screws. The ends of the device may be changed so as to provide more surface contact area thereby reducing the surface pressure on the bone. Bellows are employed in many different types of thermal controllers so that the germane design equations are not presented here. The use of bellows for the treatment of idiopathic scoliosis as described in this patent represents a unique application not cited in other patents with the restriction that the material must be compatible with body fluids as contrasted with the materials normally employed in patents involving thermal controllers and other pressure type applications.

An additional feature is shown in the embodiment of this invention in FIG. 7. With the two separate flexible bladders, that may be employed with mechanical springs (not shown), the bladders exclude flow of body fluids into and out of the main cylinder 70 of this invention as the total volume of the expansion of one bladder is compensated by the volume contraction of the other bladder. The pressures initially applied to the bladders, 71 and 74, will control and determine either the extension or contraction configuration of the device. The only change in the displaced body fluid arises from the displacement of the shaft 73 as it moves in and out of the cylinder 70.

The use of a single flexible bladder with a mechanical spring will minimize the flow of body fluids in the configuration of FIG. 7. However, as noted in the calculation given above for a typical small spring, the total size of this invention is relatively small and the total flexing, as given by the calculation above, is also small so that double bladders, as illustrated in FIG. 7, may not be imperative for many applications. As with the other embodiments, the attachment means to the spinal column pedicles will be by screws (not shown) through the holes 75, of FIG. 7.

FIG. 8 is a cross section of another embodiment of this invention, which makes use of “wave springs”. Wave springs offer certain design advantages over the more conventional helical springs including stability and relative size for the equivalent displacement and force of the more common helical springs. This particular illustration offers one design that can function as either an extraction or compression configuration. With one wave spring attached to the movable cap 88 and another wave spring attached to the fixed head 82, the device acts in the contraction configuration with the forces F1 and F2 directed as shown. By reversing the direction of the forces F1 and F2, this same device will act in the compression configuration. Because of this ability to act in both configurations, this embodiment is very optimal. As described and pictured in FIG. 8, one can visualize that the individual wave springs, such as 83 and 84, are welded at each of the respective points of contact of one spring relative to the adjacent one. This is necessary so that they may function whether being compressed or being stretched, one with respect to the other. This concept of joining adjacent wave spring elements is new and is not generally accounted for by the manufacturers of wave springs. Manufacturers wish to have slipping at the contact points of one wave spring with respect to the other. By welding or otherwise fastening these contact points, one to the other, the overall stiffness of the combination becomes greater. This implies that they spring rate of said welded wave springs will be much larger than the non-welded units.

As described before, the movable element 89 of FIG. 8 is threaded and matches the threaded movable cap 88. Further, the end of the threaded movable element 89 is “upset” in such a manner that will prevent the movable element 89 from being unscrewed completely or detached from the movable element 88. This upsetting of the end will prevent the surgeon from “accidentally” opening the unit too far and disconnecting the movable elements from the head 82. As noted before, the wave spring elements will not have to be large as the force levels required will be small and the stiffness will be increased by the joining of the several elements at their contact regions. This will also afford the designer the option to employ plastic wave springs instead of metallic wave springs as the total force levels need be one or two pounds.

As with other embodiments, the ring 88 of FIG. 8 is threaded such that the movable head 89 may be adjusted in length by rotating the head with respect to the ring 88. This adjustable length of the overall configuration will afford the surgeon means for proper alignment of the configuration to the vertebral bodies. Further, this adjustment means will afford the surgeon the control of the preload for either the compression or the extraction configuration. This preload adjustment means affords the surgeon an opportunity to visually change the effective curvature of the spine by the combination of more than one configuration being changed length-wise and through the adjustment of the preloads for each configuration.

The spring rate for the device of FIG. 8 is established by the number of waves, the sizes (inside and outside diameters) of the waves, the thickness of the material composing the individual elements of the waves, and the modulus of elasticity of the material from which the waves are made. As can be understood by anyone versed in the art of mechanics, the combination of the properties just noted plus the adjustability by the rotation of the movable head 89 relative to the ring 88 permits a wide variety of spring rates and physical lengths of this device. And as described previously, this device may be used by attachment to pedicles and/or transverse processes of the vertebral bodies.

As many possible embodiments may be made of the invention without departing from the scope thereof, it is to be understood that all matter herein set forth and shown in the accompanying drawings is to be interpreted as illustrative and not in a limiting sense.

1. A device for providing surgical treatment of idiopathic scoliosis wherein said device includes means for providing variable forces to vertebral bodies of human spines comprising a preloaded spring or series of preloaded springs; attachment means for attaching said springs to pedicles of said vertebral bodies; said spring or springs providing small variable forces to supplant and counter the unbalance of affected muscles of a patient with scoliosis while allowing general flexing of the spinal column; said small forces being provided over an extended period of time to provide a gradual yielding, stretching and relaxation of the affected muscles without inducing any lower back pain; and said variable forces being determined and set in the initial device design and the initial preload of the springs.

2. A device as in claim 1 wherein said means for providing variables forces comprises a series of springs, screws and or wires for attaching said springs to the transverse processes of said vertebral bodies wherein the springs exert variable forces as said vertebral bodies move through general flexing of the spinal column, said variable forces initially established by the initial device design and the initial preload of the springs and said variable forces being controlled by the surgeon through adjustment of the overall length of said device.

3. A device as in claim 1 wherein said means for providing variable forces comprise a series of wave springs, said variable forces initially established by the initial design and the initial preload of the springs to act in the compression mode.

4. A device as in claim 1 wherein said means for providing variable forces comprise a series of wave springs, said variable forces initially established by the initial device design and the initial preload of the springs, said initial force either acting in and extraction mode or a compression mode as the individual wave spring elements are fastened together at their respective contacting regions.

5. A device as in claim 1 wherein said means for providing variable forces comprises one or a series of bladders, and said variable forces are initially established by the initial device design and the initial prescribed pressures in the bladders.

6. A device for providing surgical treatment of idiopathic scoliosis wherein said device includes means for providing variable forces to vertebral bodies comprising a bladder and one or more springs, screws for attaching said variable forces to the pedicles of vertebral bodies, wherein said springs through the attachment means exert variable forces as the vertebral bodies move through general flexing of the spinal column, said variable forces being initially established by the initial force design and initial preload of said springs, said bladder being arranged to minimize the displaced volume of the body of the device as the movable elements depose the surrounding body fluids.

7. A device as in claim 1 wherein said means for providing variable forces comprise a series of wave springs, said variable forces being initially established by the initial device design and the initial preload of the springs, said initial force either acting in an extraction mode or a compression mode.

8. A device as in claim 1 wherein the means for providing the variable forces comprise individual wave springs of metallic or plastic material, means for attaching said wave springs to one another at their respective contact points or surfaces thereby permitting forces to be transmitted through said series of springs in either a compression mode and or a tension mode through said means of attachment to act on the pedicles of vertebral bodies as a corrective series of forces to promote alignment of the spine.

9. A device as in claim 1 wherein the spring material may be metallic such as stainless steel or a human body compatible plastic.

10. A device comprising a series of metallic or plastic bellows, means for attachment to the pedicles or the transverse process is of vertebral bodies of the human spin by which variable forces ar applied to said vertebral bodies of the spin, with said forces acting to strain the muscles and tendons to align the continuum of the vertebral bodies in a proper orientation.

11. A device as in claim 1 wherein said variable force is in the range of one (1) to two (2) pounds.
